(19) United States
(12) Patent Application Publication
(10) Pub. No.: US 2025/0084397 A1
(43) Pub. Date: Mar. 13, 2025

(54) METHODS OF PURIFYING DNA FOR GENE SYNTHESIS

(71) Applicant: MODERNATX, INC., Cambridge, MA (US)

(72) Inventor: Kevin Smith, Cambridge, MA (US)

(73) Assignee: ModernaTX, Inc., Cambridge, MA (US)

(21) Appl. No.: 18/725,999

(22) PCT Filed: Nov. 4, 2022
(86) PCT No.: PCT/US2022/048904

§ 371 Date: Jul. 1, 2024

Related U.S. Application Data
(60) Provisional application No. 63/296,450, filed on Jan. 4, 2022.

(51) Int. Cl. C12N 15/10; C12N 9/22

(52) U.S. Cl. CPC C12N 15/1013; C12N 9/22

(57) ABSTRACT

Provided herein are methods of purifying nucleic acids (e.g., DNA) for gene synthesis using combinations of nucleases. Also provided are improved products for use in the production of RNA.

### RELATED APPLICATIONS

This application claims the benefit under 35 U.S.C. 119(e) of U.S. provisional application No. 63/296,450 filed Jan. 4, 2022 which is incorporated by reference herein in its entirety.

### GOVERNMENT LICENSE RIGHTS

This invention was made with Government support under Agreement No. HRO011-20-9-0118, awarded by DARPA. The Government has certain rights in the invention.

### BACKGROUND

Messenger RNA (mRNA) is an emerging alternative to conventional small molecule and protein therapeutics due to the potency and programmability of mRNA. mRNA encoding a desired therapeutic protein can be administered to a subject for in vivo expression of the protein to generate a therapeutic effect, such as vaccination or replacement of a protein encoded by a mutated gene. In vitro transcription (IVT) of a DNA template using a bacteriophage RNA polymerase is a useful method of producing mRNAs for therapeutic applications. The process requires high quality DNA template to achieve quality commercial scale mRNA.

One method for producing DNA template for IVT involves gene synthesis, a process of assembling gene-length fragments from shorter groups of oligonucleotides. In order to enhance the integrity of IVT and the resultant mRNA product, it is desirable to limit sequence errors in the DNA template. Existing sequence error correction methods have demonstrated that it is possible to ameliorate some sequence errors during gene synthesis. The effectiveness of any error correction can be determined using, for instance, next-generation sequencing (NGS).

### SUMMARY

Provided herein are methods of purifying nucleic acids, such as DNA, using nuclease digestion processes.

In some aspects a method for processing a DNA, by preparing a sample of heteroduplex DNA, wherein at least one heteroduplex DNA in the sample comprises a mismatch DNA having one or more sequence errors, performing a dual nuclease digestion on the sample to produce a digested product by contacting the sample with an endonuclease, wherein the endonuclease is T7E1 endonuclease to cleave the mismatch DNA at the sequence error site to produce one or more DNA fragments and contacting the sample with a second nuclease, wherein the second nuclease is a mung bean nuclease to further degrade the DNA fragments, thereby producing a purified sample of heteroduplex DNA is provided.

In some embodiments, the purified sample of heteroduplex DNA produced by the method has error-rate reductions of 15-60% relative to a comparable method performed without digestion with mung bean nuclease. In some embodiments, the purified sample of heteroduplex DNA produced by the method has error-rate reductions of 20-30% relative to a comparable method performed without digestion with mung bean nuclease.

In some embodiments, less than 5% of total nucleic acid in the purified sample of heteroduplex DNA is comprised of mismatched DNA and DNA fragments. In some embodiments, at least 99% of heteroduplex DNA has 100% base complementarity and wherein at least 99% of the heteroduplex DNA is full length.

In some embodiments, a re-assembly PCR step is performed following nuclease digestion on the digested product, thereby producing a purified sample of DNA template. In some embodiments, a purification step is performed following re assembly PCR. In some embodiments, the purification step is a solid-phase reversible immobilization (SPRI) paramagnetic bead process.

In some embodiments, a purification step is not performed between the nuclease digestion and the re assembly PCR.

In some embodiments, the digested product is used in re assembly step at a maximum volume of 50 μL.

In some embodiments, the sample is contacted with both T7E1 endonuclease and mung bean nuclease at the same time.

In some embodiments, the sample comprises 1:1 ratio of both T7E1 endonuclease and mung bean nuclease.

In some embodiments, the dual nuclease digestion step is performed at least two times.

In some embodiments, the process is a commercial batch process.

In some embodiments, the sequence error comprises a substitution, deletion or insertion of between 1 and 10 nucleotides.

In some embodiments, the method further comprises producing mRNA with the purified sample of heteroduplex DNA.

In other aspects a purified sample of DNA template comprising, consisting of, or consisting essentially of a plurality of heteroduplex DNA, wherein at least 99% of the heteroduplex DNA has 100% base complementarity and wherein at least 99% of the heteroduplex DNA is full length is provided.

In yet other aspects a purified sample of DNA template, comprising, consisting of, or consisting essentially of a plurality of DNA template, wherein at least 99% of the DNA template has 100% base complementarity and wherein at least 99% of the DNA template is full length.

A composition comprising, consisting of, or consisting essentially of a heteroduplex DNA comprising, consisting of, or consisting essentially of a mismatch DNA having one or more sequence errors, T7E1 endonuclease and mung bean nuclease is provided in other aspects.

A composition comprising, consisting of, or consisting essentially of a plurality of heteroduplex DNA, T7E1 endonuclease and mung bean nuclease, wherein at least 90-100% of the heteroduplex DNA is full length is provided in other aspects. In some embodiments, one endonuclease is T7E1 endonuclease and the other endonuclease is mung bean nuclease.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE DRAWINGS

FIG. 1 shows efficiency of error correction in PCR products as a result of nuclease treatments. The graph depicts quantification of error-rate removal in PCR products as a result of digestion of four DNA template samples (DNA Fragments 1-4) with T7E1 and mung bean nucleases prior to gene synthesis. Samples labeled “T7&MungBean_mixture” and “T7&MungBean_stepwise” indicate samples that were co-digested with both nucleases concurrently and samples first digested with T7E1 alone before adding mung bean nuclease, respectively. A positive control (T7 digestion followed by a SPRI purification step, “T7+S”) and negative control (Contr) were included. Bars from left to right correspond to Contr, T7+S, T7&MungBean_mixture, T7&MungBean(d3)_mixture_T7&MungBen(d10)_mixture, T7&MungBean_stepwise, T7&MungBean(d3)_stepwise, and T7&MungBean(d10)_stepwise, respectively.

### DETAILED DESCRIPTION

The present disclosure relates to methods of error correction during gene synthesis, for a downstream in vitro transcription (IVT) reaction. Gene synthesis involves assembly of many oligonucleotides into a single larger piece of DNA. There are a number of methods for gene synthesis including polymerase-based assembly methods. The quality and integrity as well as the yield are important factors that go into the selection of an appropriate gene synthesis method. Several factors can influence the quality of the synthesized gene product. For instance, the quality of the reagents and materials used, the methods, and the purification steps can influence the quality of the synthesized gene product. Without further steps to mitigate errors in the process, size purity in the template sample is greatly diminished in some instances.

Some methods for reducing error rate post-synthesis include size selection methods such as high-performance liquid chromatography (HPLC) or polyacrylamide gel electrophoresis (PAGE) to filter truncated sequences, hybridization-selection techniques, sequencing-based retrieval methods, and protein/enzymatic error correction. Each method has some drawbacks. For instance, size separation methods are both labor-intensive and ineffective against small errors such as single-base deletions, insertions or substitutions.

An aspect of the instant disclosure relates to a new more efficient method for significantly enhancing error correction during gene synthesis, which results in the production of high-quality DNA. The method involves, in some aspects, preparing a sample of heteroduplex DNA and treating the heteroduplex DNA with a combination of T7E1 endonuclease and mung bean nuclease.

Initially, the sample of heteroduplex DNA can be prepared. A heteroduplex refers to a double stranded nucleic acid molecule having a target sequence (i.e., the sequence of a gene of interest or fragments thereof, which is being synthesized), wherein each strand of the nucleic acid is derived from a different parent molecule. Typically, the sample may be prepared by generating sets of complementary oligonucleotides and combining the oligonucleotides under conditions that allow the complementary oligonucleotide strands to hybridize to one another. In some instances, the oligonucleotides hybridize to form a heteroduplex DNA having 100% or perfect complementarity. However, some of the oligonucleotides form hybrids having less than perfect complementarity. These heteroduplex DNA comprise one or more mismatched bases and are referred to as mismatch DNA having one or more sequence errors. A sequence error In some embodiments, is a single-base deletion and/or mismatch such as a substitution or insertion. The mismatch can comprise anywhere from 1 to at least 12 nucleotides, such as a mismatch of at least 1, 2, 3, 4, 5, 6, 7, 8, 9, 10, 11, or 12 nucleotides.

A sequence error refers to any change in the nucleotide sequence of a nucleic acid molecule that is different from the desired target sequence for the nucleic acid molecule. The sequence error can be a substitution, insertion, or deletion in the sequence.

In some embodiments, at least one of the mismatched DNA having one or more sequence errors have 1, 2, 3, 4, 5, 6, 7, 8, 9, 10, 11, 12, 13, 14, or 15 sequence errors.

In some embodiments, the mismatch DNA having one or more sequence errors have less than 100%, such as less than or equal to 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, or 50% complementarity. The strands of a double-stranded molecule may have partial, substantial or full complementarity to each other and will form a duplex hybrid. The term “complementarity” describes the capacity for Watson-Crick base-pairing of nucleosides/nucleotides. Watson-Crick base pairs are guanine (G)-cytosine (C) and adenine (A)-thymine (T)/uracil (U). Nucleic acids also comprise nucleosides with modified nucleobases, for example 5-methyl cytosine may be used in place of cytosine. The term complementarity encompasses Watson Crick base-paring between non-modified and modified nucleobases.

Percent complementary refers to the proportion of nucleotides (in percent) of a contiguous nucleotide sequence in a nucleic acid molecule which across the contiguous nucleotide sequence are complementary to a reference sequence. The percentage of complementarity may be calculated by counting the number of aligned nucleobases that are complementary between the two sequences (when aligned with the target sequence 5′-3′ and the reference sequence from 3′-5′), dividing that number by the total number of nucleotides in the target sequence and multiplying by 100. In such a comparison a nucleobase/nucleotide which does not align or form a base pair is termed a mismatch.

The sample of heteroduplex DNA can be treated with an endonuclease. In some embodiments, the endonuclease recognizes the distortions in the DNA helix of the heteroduplex that are caused by mis-hybridized bases on either strand, or sequence errors. The endonuclease can cleave at or near the recognized site, causing the production of two DNA fragments. In some embodiments, the endonuclease is selected from the group consisting of T7 endonuclease I, (T7E1), Cel-I, Surveyor, T4 Endonuclease VII, Deoxyribonuclease I (DNase I), RecBCD endonuclease, Bal 31 endonuclease, endonuclease I (endo I), Endonuclease II, Neurospora endonuclease, S1-nuclease, P1-nuclease, AP endonuclease, and Endo R. In some embodiments, the endonuclease is T7E1. In some embodiments, the amount of endonuclease (e.g., T7E1) used in the reaction is about 0.1 μL, 0.2 μL, 0.3 μL, 0.4 μL, 0.5 μL, 0.6 μL, 0.7 μL, 0.8 μL, 0.9 μL, 1.0 μL, 1.1 μL, 1.2 μL, 1.3 μL, 1.4 μL, 1.5 μL, 1.6 μL, 1.7 μL, 1.8 μL, 1.9 μL, 2.0 μL, 2.1 μL, 2.2 μL, 2.3 μL, 2.4 μL, 2.5 μL, 2.6 μL, 2.7 μL, 2.8 μL, 2.9 μL, 3.0 μL, 3.1 μL, 3.2 μL, 3.3 μL, 3.4 μL, 3.5 μL, 3.6 μL, 3.7 μL, 3.8 μL, 3.9 μL or 4.0 μL. In some embodiments, the amount of endonuclease used in the reaction is about 2.0 μL of T7E1.

The endonuclease can cleave the mismatch DNA at or near the sequence error site to produce one or more DNA fragments. For instance, T7E1 cleaves 5′ of a detected sequence mismatch, producing DNA fragments having an exposed 5′ phosphate group on both strands. The DNA fragments can be contacted with mung bean nuclease in order to further degrade the DNA fragments. Mung bean nuclease is useful, for instance, in removing 3′ and 5′ extensions or single-stranded regions from DNA termini. Thus, sequential nuclease activity can be used to cleave the exposed nucleotides of the error-containing region of the DNA fragments left over by the mismatch cleaving enzymes. The mung bean nuclease digestion can remove the DNA fragments, leaving a sample of DNA with a significantly improved error rate. In some embodiments, the endonuclease digestion step and/or mung bean nuclease digestion step may be repeated one, two, three, four, five or more times to further reduce the presence of errors in the heteroduplex DNA. In some embodiments, the amount of mung bean nuclease used in the reaction is about 0.1 μL, 0.2 μL, 0.3 μL, 0.4 μL, 0.5 μL, 0.6 μL, 0.7 μL, 0.8 μL, 0.9 μL, 1.0 μL, 1.1 μL, 1.2 μL, 1.3 μL, 1.4 μL, 1.5 μL, 1.6 μL, 1.7 μL, 1.8 μL, 1.9 μL, 2.0 μL, 2.1 μL, 2.2 μL, 2.3 μL, 2.4 μL, 2.5 μL, 2.6 μL, 2.7 μL, 2.8 μL, 2.9 μL, 3.0 μL, 3.1 μL, 3.2 μL, 3.3 μL, 3.4 μL, 3.5 μL, 3.6 μL, 3.7 μL, 3.8 μL, 3.9 μL or 4.0 μL. In some embodiments, about 0.5-10 units of Mung Bean Nuclease per g DNA of a mung bean nuclease stock, i.e., of about 5-50 U/μL is used. In some embodiments, about 1 unit of Mung Bean Nuclease per g DNA of a mung bean nuclease stock, i.e., of about 10 U/μL (10,000 U/mL) is used. In some embodiments, the amount of mung bean nuclease used in the reaction is about 2.0 μL of mung bean nuclease. In some embodiments, mung bean nuclease is taken from its stock solution (at about 5-50 U/μL, e.g., about 10 U/μL) prior to contacting mung bean nuclease with DNA substrate. In some embodiments, stock solutions of mung bean nuclease are diluted to dilutions of about 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20, or in ranges between any of the aforementioned dilutions, prior to contacting mung bean nuclease with DNA substrate.

In some embodiments, a composition comprising, consisting of, or consisting essentially of a plurality of DNA template, a T7E1 endonuclease, and a mung bean endonuclease. In embodiments, the heteroduplex DNA of the composition may comprise a mismatch DNA having one or more sequence errors. In embodiments, the heteroduplex DNA of the composition may be at least 90-100% full length. In embodiments, the composition may comprise a volume of T7E1 endonuclease of about 0.0.1 μL, 0.2 μL, 0.3 μL, 0.4 μL, 0.5 μL, 0.6 μL, 0.7 μL, 0.8 μL, 0.9 μL, 1.0 μL, 1.1 μL, 1.2 μL, 1.3 μL, 1.4 μL, 1.5 μL, 1.6 μL, 1.7 μL, 1.8 μL, 1.9 μL, 2.0 μL, 2.1 μL, 2.2 μL, 2.3 μL, 2.4 μL, 2.5 μL, 2.6 μL, 2.7 μL, 2.8 μL, 2.9 μL, 3.0 μL, 3.1 μL, 3.2 μL, 3.3 μL, 3.4 μL, 3.5 μL, 3.6 μL, 3.7 μL, 3.8 μL, 3.9 μL or 4.0 μL. In embodiments, the composition may comprise a volume of mung bean nuclease of about 0.0.1 μL, 0.2 μL, 0.3 μL, 0.4 μL, 0.5 μL, 0.6 μL, 0.7 μL, 0.8 μL, 0.9 μL, 1.0 μL, L1 μL, 1.2 μL, 1.3 μL, 1.4 μL, 1.5 μL, 1.6 μL, 1.7 μL, 1.8 μL, 1.9 μL, 2.0 μL, 2.1 μL, 2.2 μL, 2.3 μL, 2.4 μL, 2.5 μL, 2.6 μL, 2.7 μL, 2.8 μL, 2.9 μL, 3.0 μL, 3.1 μL, 3.2 μL, 3.3 μL, 3.4 μL, 3.5 μL, 3.6 μL, 3.7 μL, 3.8 μL, 3.9 μL or 4.0 μL or in ranges between any of the aforementioned volumes.

In some embodiments, the DNA is contacted with a solution that contains both the endonuclease and mung bean nuclease. The samples can be incubated with the T7E1 endonuclease and mung bean nuclease under conditions for optimal nuclease activity. In some embodiments, the samples are incubated with both nucleases at a temperature of about 35-55° C. The reaction may also be allowed to proceed for an optimal time determined by the particular nuclease being used. Typically, the length of the reaction is 10-60 minutes, and preferably for about 45 minutes. In some embodiments, the reaction is performed at 30° C. or 37° C. for about 30 minutes. Following the incubation with nuclease the reactions may optionally be stopped by a stop mechanism. For instance, the reaction may be terminated using heat inactivation. Heat inactivation can be achieved by raising the temperature of the reaction to a temperature above 55° C. for a period of time, such as 5 minutes or more. In some embodiments, the reaction may be heat inactivated by raising the temperature to 70° C.-80° C., optimally 75° C. for 5-15 minutes, optimally 10 minutes.

In some embodiments, the samples are incubated with endonuclease first and then Mung Bean nuclease is added later (e.g., about 5 minutes, 10 minutes, 15 minutes, 20 minutes, 25 minutes, 30 minutes, 45 minutes, 60 minutes, or 90 minutes or more after addition of the endonuclease) and the mixture is further incubated. In some embodiments, the endonuclease may be added to the heteroduplex DNA sample first and the reaction allowed to proceed to completion. Subsequently the mung bean nuclease may be added to the sample. In other embodiments the endonuclease and the mung bean nuclease may be added to the sample at the same time.

The efficiency of the reaction can depend to some extent on the relative amounts of nucleases and heteroduplex DNA in the sample. The relative amounts of both nucleases may be considered as an optimal ratio. In some embodiments, the ratio of the endonuclease (e.g., T7E1 endonuclease) and mung bean nucleases may be about 20:1, 15:1, 10:1, 5:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, or 1:100. In some embodiments, the T7E1 endonuclease and mung bean nuclease are in a ratio of about 1:1.

Also provided are compositions comprising endonuclease (e.g., T7E1 endonuclease) and mung bean. In some embodiments, the ratio of the endonuclease (e.g., T7E1 endonuclease) and mung bean nucleases is about 20:1, 15:1, 10:1, 5:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, or 1:100. In some embodiments, the T7E1 endonuclease and mung bean nuclease are in a ratio of about 1:1.

The efficiency of the reaction may also depend, in some embodiments, on the amount of heteroduplex DNA being processed. For instance, the reaction volume may play a role in the efficiency of the error correction reaction. Separately, the concentration of heteroduplex DNA can also impact the digestion and error correction efficiency in the reaction.

After the second nuclease reaction or combined nuclease reaction is complete the heteroduplex DNA can be joined together to form a sample of DNA template using a PCR re-assembly and final purification steps. PCR re-assembly can be performed using methods and conditions known in the art. Briefly, an exemplary process involves a pre-assembly step where the oligonucleotides are mixed with PCR components and subjected to temperature cycling. Following the final extension step, mixtures of template, forward and reverse amplification primers flanking the outer oligonucleotides of each construct are cycled and then a final elongation step can be performed. Basic methods for PCR re-assembly are described, for instance, in US 2008/0182296, Wu et al (J Biotechnol. 2006 Jul. 25; 124(3):496-503.) and Sequeira et al (BMC Biotechnology volume 16, Article number: 86 (2016)) and are available commercially from a number of sources.

Following re-assembly PCR, the DNA samples may be purified to remove any of the components involved in the assay. Multiple purification methods are known in the art and could be applied. For instance, solid phase reverse immobilization (SPRI) may be used. SPRI involves the use of paramagnetic beads, typically made of polystyrene surrounded by a layer of magnetite, which is coated with carboxyl molecules. The beads reversibly bind to DNA in the presence of a binding agent such as polyethylene glycol (PEG) and salt. The PEG causes the negatively charged DNA to bind to the carboxyl groups on the bead surface. The concentration of PEG and salt in the reaction and the volumetric ratio of beads to DNA can be adjusted to influence the immobilization. In some embodiments, a range of PEG of 15% to 20% is used. In other embodiments 15% or 20% PEG is used. SPRI beads are commercially available, for instance, from Beckman. SPRI is particularly useful because of its ability to be used in automated systems.

In some embodiments, the removal of errors from a DNA provides a purified sample of DNA template, wherein a larger proportion of the DNA comprise the correct sequence relative to prior art methods. For example, the purified sample of DNA template produced as disclosed herein may have an error frequency that is reduced by 1.5, 2, 2.5, 3, 3.5, 4, 4.5, 5 or more fold relative to a product produced using only single endonuclease digestion. In some embodiments, DNA template produced may have an error frequency that is reduced by 1.5, 2, 2.5, 3, 3.5, 4, 4.5, 5, 6, 7, 8, 9, or 10 or more fold relative to a product produced using a method without error correction.

An error rate can be determined for a sample of heteroduplex DNA. The error rate may be determined as the number of errors detected at a given base, divided by the total number of sequencing reads in the sample. Error rates can be further separated by the specific error sub-type if desired. In some embodiments, the purified sample of DNA template produced by the method has error-rate reductions of 5-50%, 5-40%, 5-30%, 5-20%, 5-15%, 5-10%, 10-50%, 10-40%, 10-30%, 10-20%, 10-15%, 15-50%, 15-40%, 15-30%, 15-20%, 15-18%, 20-50%, 20-40%, or 20-30% relative to a comparable method performed without second nuclease digestion. In some embodiments, the purified sample of DNA template produced by the method has error-rate reductions of 5-50%, 5-40%, 5-30%, 5-20%, 5-15%, 5-10%, 10-50%, 10-40%, 10-30%, 10-20%, 10-15%, 15-50%, 15-40%, 15-30%, 15-20%, 15-18%, 20-50%, 20-40%, or 20-30% relative to a product produced using a method without error correction.

Thus, In some embodiments, a DNA product having very low levels to no levels of sequence errors can be produced according to the methods disclosed herein. A composition comprising heteroduplex DNA (i.e. DNA before PCR re-assembly) processed according to these methods has, in some embodiments, a total nucleic acid content, wherein less than 5% of the total nucleic acid in the heteroduplex DNA sample is comprised of mismatched DNA and DNA fragments. In some embodiments, less than 4%, less than 3.5%, less than 3%, less than 2.5%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.7%, less than 0.6%, less than 0.5%, less than 0.4%, less than 0.3%, less than 0.2%, or less than 0.1% of the total nucleic acid in the heteroduplex DNA sample is comprised of mismatched DNA and DNA fragments. In some embodiments, the heteroduplex DNA sample is free of mismatched DNA and DNA fragments and thus has 0% mismatched DNA and DNA fragments.

A composition processed according to these methods may also be a sample of heteroduplex DNA, wherein at least 99% of the heteroduplex DNA has 100% base complementarity and wherein at least 99% of the heteroduplex DNA is full length. In some embodiments, at least 99.1%, at least 99.2%, at least 99.3%, at least 99.4%, at least 99.5%, at least 99.6%, at least 99.7%, at least 99.8%, at least 99.9% or 100% of the DNA template has 100% base complementarity and wherein at least 99.1%, at least 99.2%, at least 99.3%, at least 99.4%, at least 99.5%, at least 99.6%, at least 99.7%, at least 99.8%, at least 99.9% or 100% of the heteroduplex DNA is full length. In some embodiments, the heteroduplex DNA has 100% base complementarity and 100% of the heteroduplex DNA is full length.

A composition comprising DNA template (i.e. DNA template after PCR re-assembly) processed according to these methods has, in some embodiments, a total nucleic acid content wherein less than 5% of the total nucleic acid is comprised of mismatched DNA and DNA fragments. In some embodiments, less than 4%, less than 3.5%, less than 3%, less than 2.5%, less than 2%, less than 1.9%, less than 1.8%, less than 1.7%, less than 1.6%, less than 1.5%, less than 1.4%, less than 1.3%, less than 1.2%, less than 1.1%, less than 1%, less than 0.9%, less than 0.8%, less than 0.7%, less than 0.6%, less than 0.5%, less than 0.4%, less than 0.3%, less than 0.2%, or less than 0.1% of the total nucleic acid in the DNA template is comprised of mismatched DNA and DNA fragments. In some embodiments, the DNA template is free of mismatched DNA and DNA fragments and thus has 0% mismatched DNA and DNA fragments.

A composition processed according to these methods may also be a sample of DNA template wherein at least 99% of the DNA template has 100% base complementarity and wherein at least 99% of the DNA template is full length. In some embodiments, at least 99.1%, at least 99.2%, at least 99.3%, at least 99.4%, at least 99.5%, at least 99.6%, at least 99.7%, at least 99.8%, at least 99.9% or 100% of the DNA template has 100% base complementarity and wherein at least 99.1%, at least 99.2%, at least 99.3%, at least 99.4%, at least 99.5%, at least 99.6%, at least 99.7%, at least 99.8%, at least 99.9% or 100% of the DNA template is full length.

In some embodiments, the DNA template has 100% base complementarity and 100% of the DNA template is full length.

In some embodiments, nuclease digestion may excessively fragment the DNA if not attenuated through heat inactivation. Thus, in some embodiments, the PCR re-assembly step may be performed immediately after the nuclease treatment, without any further processing. In addition to reducing labor and costs, this advantage also supports the ability to automate the process, which allows for enhanced benefits in commercial development of mRNA therapeutics and vaccines.

The methods disclosed herein may be automated. Thus, the whole process involving the steps of oligonucleotide synthesis, heteroduplex formation, dual nuclease treatment with T7E1 endonuclease and mung bean nuclease, PCR re-assembly, and optionally final purification, e.g., SPRI may be preprogrammed and fully automated for large scale development of DNA template.

In some embodiments, the nuclease digestion compositions and methods of the present disclosure may be used for laboratory scale preparations of nucleic acid templates (e.g., preparing samples of nucleic acids with a total volume that is measured in microliters or milliliters including nucleic acid solutions handled and treated in containers such as microtubes (of about 200 μL or less), Eppendorf tubes (of about 0.5-2.0 mL), or conical tubes (of about 3-100 mL). In some embodiments, the nuclease digestion compositions and methods of the present disclosure may be used for industrial scale preparation of nucleic acid templates involving commercial batch processes (e.g., preparing samples of nucleic acids with a total volume that is measured in liters such as those that are handled and treated in an automated fashion in large containers or vats with a total volume of about 1, 5, 25, 100, 200, 300, 400, 500, or more liters).

### Nucleic Acids

Aspects of the present disclosure relate to compositions comprising nucleic acids and methods of producing nucleic acids. As used herein, the term “nucleic acid” includes multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). The term nucleic acid includes polyribonucleotides as well as polydeoxyribonucleotides. The term nucleic acid also includes polynucleosides (i.e., a polynucleotide minus the phosphate) and any other organic base containing polymer. Non-limiting examples of nucleic acids include chromosomes, vectors, plasmids, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. A nucleic acid (e.g., mRNA) may include a substitution and/or modification. In some embodiments, the substitution and/or modification is in one or more bases and/or sugars. For example, in some embodiments a nucleic acid (e.g., mRNA) includes nucleotides having an organic group, such as a methyl group, attached to a nucleic acid base at the N6 position. Thus, in some embodiments, an mRNA includes one or more N6-methyladenosine nucleotides. A phosphate, sugar, or nucleic acid base of a nucleotide may also be substituted for another phosphate, sugar, or nucleic acid base. For example, a uridine base may be substituted for a pseudouridine base, in which the uracil base is attached to the sugar by a carbon-carbon bond rather than a nitrogen-carbon bond. Thus, in some embodiments, a nucleic acid (e.g., mRNA) is heterogeneous in backbone composition thereby containing any possible combination of polymer units linked together such as peptide-nucleic acids (which have an amino acid backbone with nucleic acid bases).

The nucleic acid sequences of the present invention include nucleic acid sequences that have been removed from their naturally occurring environment and engineered nucleic acids. An “engineered nucleic acid” is a nucleic acid that does not occur in nature. It should be understood, however, that while an engineered nucleic acid as a whole is not naturally occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence.

Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

In some embodiments, a nucleic acid is present in (or on) a vector. Examples of vectors include but are not limited to bacterial plasmids, phage, cosmids, phasmids, fosmids, bacterial artificial chromosomes, yeast artificial chromosomes, viruses and retroviruses (for example vaccinia, adenovirus, adeno-associated virus, lentivirus, herpes-simplex virus, Epstein-Barr virus, fowlpox virus, pseudorabies, baculovirus) and vectors derived therefrom. In some embodiments, a nucleic acid (e.g., DNA) used as an input molecule for in vitro transcription (IVT) is present in a plasmid vector.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators) and is in a form suitable for use within genetically engineered protein production systems. Such isolated molecules are those that are separated from their natural environment.

In some embodiments, a nucleic acid is a DNA template for IVT. An “in vitro transcription template” (IVT template), or “DNA template” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

In some embodiments, the DNA template may be incorporated within a nucleic acid vector, which may be a circular nucleic acid such as a plasmid. In other embodiments it is a linearized DNA.

A DNA template may include an insert which may be an expression cassette or open reading frame (ORF). An “open reading frame” is a continuous stretch of DNA beginning with a start codon (e.g., methionine (ATG)), and ending with a stop codon (e.g., TAA, TAG or TGA) and encodes a protein or peptide (e.g., a therapeutic protein or therapeutic peptide). In some embodiments, an expression cassette encodes an RNA including at least the following elements: a 5′ untranslated region, an open reading frame region encoding the mRNA, a 3′ untranslated region and a polyA tail. The open reading frame may encode any mRNA sequence, or portion thereof. The DNA may be single-stranded or double-stranded. In some embodiments, the DNA is present on a plasmid or other vector. A DNA may include a polynucleotide encoding a polypeptide of interest. A DNA, in some embodiments, includes an RNA polymerase promoter (e.g., a T7 RNA polymerase promoter) located 5′ from and operably linked to a polynucleotide encoding a polypeptide of interest.

The length of the DNA, and thus the length of the RNA of interest which it encodes, may vary. For example, the DNA (and/or the RNA of interest) may have a length of about 200 nucleotides to about 10,000 nucleotides. In some embodiments, the DNA (and/or the RNA of interest) has a length of 200-500, 200-1000, 200-1500, 200-2000, 200-2500, 200-3000, 200-3500, 200-4000, 200-4500, 200-5000, 200-5500, 200-6000, 200-6500, 200-7000, 200-7500, 200-8000, 200-8500, 200-9000, or 200-9500 nucleotides. In some embodiments, the DNA (and/or the RNA of interest) has a length of at least 200, at least 300, at least 400, at least 500, at least 600, at least 700, at least 800, at least 900, at least 1000, at least 2000, at least 3000, at last 4000, at least 5000, at least 6000, at least 7000, at least 8000, at least 9000, or at least 10,000 nucleotides.

In some embodiments, a nucleic acid vector comprises a 5′ untranslated region (UTR). A “5′ untranslated region (UTR)” refers to a region of an mRNA that is directly upstream (i.e., 5′) from the start codon (i.e., the first codon of an mRNA transcript translated by a ribosome) that does not encode a protein or peptide. 5′ UTRs are further described herein, for example in the section entitled “Untranslated Regions”.

In some embodiments, a nucleic acid vector comprises a 3′ untranslated region (UTR). A “3′ untranslated region (UTR)” refers to a region of an mRNA that is directly downstream (i.e., 3′) from the stop codon (i.e., the codon of an mRNA transcript that signals a termination of translation) that does not encode a protein or peptide. 3′ UTRs are further described herein, for example in the section entitled “Untranslated Regions”.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

Aspects of the disclosure relate to populations of molecules. As used herein, a “population” of molecules (e.g., DNA molecules) generally refers to a preparation comprising a plurality of copies of the molecule (e.g., DNA) of interest, for example a cell extract preparation comprising a plurality of expression vectors encoding a molecule of interest (e.g., a DNA encoding an RNA of interest).

A nucleic acid (e.g., mRNA) typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally occurring nucleotides, synthetic nucleotides and modified nucleotides, unless indicated otherwise.

Examples of naturally occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m⁵UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, an RNA transcript (e.g., mRNA transcript) of the present disclosure may include a modified nucleobase selected from pseudouridine (ψ), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, an RNA transcript (e.g., mRNA transcript) includes a combination of at least two (e.g., 2, 3, 4 or more) of the foregoing modified nucleobases.

### In Vitro Transcription

The purified DNA template produced using the methods disclosed herein is of optimal high quality and is thus useful in the production of mRNA in an in vivo transcription (IVT) reaction. Aspects of the present disclosure provide methods of producing (e.g., synthesizing) an RNA transcript (e.g., mRNA transcript) comprising contacting a DNA template with an RNA polymerase (e.g., a T7 RNA polymerase, a T7 RNA polymerase variant, etc.) under conditions that result in the production of the RNA transcript. This process is referred to as “in vitro transcription” or “IVT”. IVT conditions typically require a purified DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction.

In some embodiments, the concentration of DNA in an IVT reaction mixture is about 0.01-0.10 mg/mL, 0.01-0.09 mg/mL, 0.01-0.075 mg/mL, 0.025-0.075 mg/mL, 0.01-0.05 mg/mL, 0.02-0.08 mg/mL, 0.02-0.06 mg/mL, 0.03-0.055 mg/mL, 0.04-0.05 mg/mL, or 0.05 mg/mL. In some embodiments, the concentration of DNA is maintained at a concentration of above 0.01 mg/mL during the entirety of an IVT reaction. In some embodiments, the concentration of DNA is maintained at a concentration is about 0.01-0.10 mg/mL, 0.01-0.09 mg/mL, 0.01-0.075 mg/mL, 0.025-0.075 mg/mL, 0.01-0.05 mg/mL, 0.02-0.08 mg/mL, 0.02-0.06 mg/mL, 0.03-0.055 mg/mL, or 0.04-0.05 mg/mL during the entirety of an IVT reaction.

In some embodiments, an IVT reaction uses an RNA polymerase selected from the group consisting of T7 RNA polymerase, T3 RNA polymerase, K11 RNA polymerase, and SP6 RNA polymerase. In some embodiments, an IVT reaction uses a T3 RNA polymerase. In some embodiments, an IVT reaction uses an SP6 RNA polymerase. In some embodiments, an IVT reaction uses a K11 RNA polymerase. In some embodiments, an IVT reaction uses a T7 RNA polymerase. In some embodiments, a wild-type T7 polymerase is used in an IVT reaction. In some embodiments, a mutant T7 polymerase is used in an IVT reaction. In some embodiments, a T7 RNA polymerase variant comprises an amino acid sequence that shares at least 50%, 60%, 70%, 80%, 90%, 95%, or 99% identity with a wild-type T7 (WT T7) polymerase. In some embodiments, the T7 polymerase variant is a T7 polymerase variant described by International Application Publication Number WO2019/036682 or WO2020/172239, the entire contents of each of which are incorporated herein by reference.

T7 RNA polymerase variants with one or more mutations relative to WT T7 RNA polymerase have several advantages in IVT reactions, including improved speed, fidelity, and reduced production of double-stranded RNA (dsRNA) transcripts. Double-stranded RNA transcripts, in which at least a portion of an RNA transcript is hybridized to another RNA molecule, elicit an innate immune response when introduced into a cell, causing degradation of both strands of a dsRNA. Minimizing the formation of dsRNA transcripts during IVT enables the production of less immunogenic, and thus more stable, RNA compositions.

The input deoxyribonucleic acid (DNA) serves as a nucleic acid template for RNA polymerase. A DNA template may include a polynucleotide encoding a polypeptide of interest (e.g., an antigenic polypeptide). A DNA template, in some embodiments, includes an RNA polymerase promoter (e.g., a T7 RNA polymerase promoter) located 5′ from and operably linked to polynucleotide encoding a polypeptide of interest. A DNA template may also include a nucleotide sequence encoding a polyadenylation (polyA) region located at the 3′ end of the gene of interest. In some embodiments, an input DNA comprises plasmid DNA (pDNA). As used herein, “plasmid DNA” or “pDNA” refers to an extrachromosomal DNA molecule that is physically separated from chromosomal DNA in a cell and can replicate independently. In some embodiments, plasmid DNA is isolated from a cell (e.g., as a plasmid DNA preparation). In some embodiments, plasmid DNA comprises an origin of replication, which may contain one or more heterologous nucleic acids, for example nucleic acids encoding therapeutic proteins that may serve as a template for RNA polymerase. Plasmid DNA may be circularized or linear (e.g., plasmid DNA that has been linearized by a restriction enzyme digest).

Some embodiments comprise performing a co-IVT reaction that includes multiple input DNAs (or populations of input DNAs). In some embodiments, each input DNA (e.g., population of input DNA molecules) in a co-IVT reaction is obtained from a different source (e.g., synthesized separately).

An RNA transcript, in some embodiments, is the product of an IVT reaction. An RNA transcript, in some embodiments, is a messenger RNA (mRNA) that includes a nucleotide sequence encoding a polypeptide of interest (e.g., a therapeutic protein or therapeutic peptide) linked to a polyA tail. In some embodiments, the mRNA is modified mRNA (mmRNA), which includes at least one modified nucleotide. In some embodiments, an RNA transcript produced by IVT is further modified by circularization, in which two non-adjacent nucleotides (e.g., 5′ and 3′ terminal nucleotides) of a linear RNA are ligated to produce a circular RNA with no terminal nucleotides.

The nucleoside triphosphates (NTPs) as provided herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The composition of NTPs in an IVT reaction may also vary. In some embodiments, each NTP in an IVT reaction is present in an equimolar amount. In some embodiments, each NTP in an IVT reaction is present in non-equimolar amounts. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. In some embodiments, the molar ratio of G:C:U:A is 2:1:0.5:1. In some embodiments, the molar ratio of G:C:U:A is 1:1:0.7:1. In some embodiments, the molar ratio of G:C:A:U is 1:1:1:1.

The same IVT reaction may include 3.75 millimolar cap analog (e.g., trinucleotide cap or tetranucleotide cap). In some embodiments, the molar ratio of the cap to any of G, C, U, or A is 1:1. In some embodiments, the molar ratio of G:C:U:A:cap is 1:1:1:0.5:0.5. In some embodiments, the molar ratio of G:C:U:A:cap is 1:1:0.5:1:0.5. In some embodiments, the molar ratio of G:C:U:A:cap is 1:0.5:1:1:0.5. In some embodiments, the molar ratio of G:C:U:A:cap is 0.5:1:1:1:0.5. In some embodiments, the amount of NTPs in a IVT reaction is calculated empirically. For example, the rate of consumption for each NTP in an IVT reaction may be empirically determined for each individual input DNA, and then balanced ratios of NTPs based on those individual NTP consumption rates may be added to a IVT comprising multiple of the input DNAs.

In some embodiments, the IVT reaction mixture comprises one or more modified nucleoside triphosphates. In some embodiments, the IVT reaction mixture comprises one or more modified nucleoside triphosphates selected from the group consisting of N6-methyladenosine triphosphate, pseudouridine (ψ) triphosphate, 1-methylpseudouridine (m¹ψ) triphosphate, 5-methoxyuridine (mo⁵U) triphosphate, 5-methylcytidine (m⁵C) triphosphate, α-thio-guanosine triphosphate, and α-thio-adenosine triphosphate. In some embodiments, the IVT reaction mixture comprises N6-methyladenosine triphosphate. In some embodiments, the IVT reaction mixture comprises pseudouridine triphosphate. In some embodiments, the IVT reaction mixture comprises 1-methylpseudouridine triphosphate. In some embodiments, the concentration of modified nucleoside triphosphates in the reaction mixture is about 0.1% to about 100%, about 0.5% to about 75%, about 1% to about 50%, or about 2% to about 25%. In some embodiments, the concentration of modified nucleoside triphosphates is about 1%, about 2%, about 3%, about 4%, about 5%, about 6%, about 7%, about 8%, about 9%, about 10%, about 15%, about 20%, or about 25%.

In some embodiments, an RNA transcript (e.g., mRNA transcript) includes a modified nucleobase selected from pseudouridine (ψ), 1-methylpseudouridine (m¹ψ), 5-methoxyuridine (mo⁵U), 5-methylcytidine (m⁵C), α-thio-guanosine and α-thio-adenosine. In some embodiments, an RNA transcript (e.g., mRNA transcript) includes a combination of at least two (e.g., 2, 3, 4 or more) of the foregoing modified nucleobases.

In some embodiments, an RNA transcript (e.g., mRNA transcript) includes pseudouridine (ψ). In some embodiments, an RNA transcript (e.g., mRNA transcript) includes 1-methylpseudouridine (m1ψ). In some embodiments, an RNA transcript (e.g., mRNA transcript) includes 5-methoxyuridine (mo⁵U). In some embodiments, an RNA transcript (e.g., mRNA transcript) includes 5-methylcytidine (m⁵C). In some embodiments, an RNA transcript (e.g., mRNA transcript) includes α-thio-guanosine. In some embodiments, an RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility represents a separate embodiment of the present invention. In some embodiments, modified nucleotides are included in an IVT mixture, and are incorporated randomly during transcription, such that the RNA contains a mixture of modified nucleotides and unmodified nucleotides.

The buffer system of an IVT reaction mixture may vary. In some embodiments, the buffer system contains Tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM.

In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:0.25, 1:0.5, 1:1, 1:2, 1:3, 1:4 or 1:5.

In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:1, 1:2, 1:3, 1:4 or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

In some embodiments, IVT methods further comprise a step of separating (e.g., purifying) in vitro transcription products (e.g., mRNA) from other reaction components. In some embodiments, the separating comprises performing chromatography on the IVT reaction mixture.

In some embodiments, the method comprises reverse phase chromatography. In some embodiments, the method comprises reverse phase column chromatography. In some embodiments, the chromatography comprises size-based (e.g., length-based) chromatography. In some embodiments, the method comprises size exclusion chromatography. In some embodiments, the chromatography comprises oligo-dT chromatography.

### Untranslated Regions

Untranslated regions (UTRs) are sections of a nucleic acid before a start codon (5′ UTR) and after a stop codon (3′ UTR) that are not translated. In some embodiments, a nucleic acid (e.g., a ribonucleic acid (RNA), e.g., a messenger RNA (mRNA)) comprising an open reading frame (ORF) encoding one or more proteins or peptides further comprises one or more UTR (e.g., a 5′ UTR or functional fragment thereof, a 3′ UTR or functional fragment thereof, or a combination thereof).

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization, and/or translation efficiency. A nucleic acid comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. 5′ UTRs also have been known to form secondary structures that are involved in elongation factor binding.

In some embodiments, UTRs are selected from a family of transcripts whose proteins share a common function, structure, feature, or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new nucleic acid.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the nucleic acids of the present disclosure as flanking regions to an ORF. This publication is incorporated by reference herein for this purpose.

Wild-type UTRs derived from any gene or mRNA can be incorporated into the nucleic acids of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, and sequences available at www.addgene.org, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the nucleic acid may comprise multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see, for example, US2010/0129877, the contents of which are incorporated herein by reference for this purpose).

The nucleic acids of the disclosure can comprise combinations of features. For example, the ORF can be flanked by a 5′ UTR that comprises a strong Kozak translational initiation signal and/or a 3 UTR comprising an oligo(dT) sequence for templated addition of a polyA tail. A 5′ UTR can comprise a first nucleic acid fragment and a second nucleic acid fragment from the same and/or different UTRs (see, e.g., US2010/0293625, herein incorporated by reference in its entirety for this purpose).

Other non-UTR sequences can be used as regions or subregions within the nucleic acids of the disclosure. For example, introns or portions of intron sequences can be incorporated into the nucleic acids of the disclosure. Incorporation of intronic sequences can increase protein production as well as nucleic acid expression levels. In some embodiments, the nucleic acid of the disclosure comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the nucleic acid comprises an IRES instead of a 5′ UTR sequence. In some embodiments, the nucleic acid comprises an IRES that is located between a 5′ UTR and an open reading frame. In some embodiments, the nucleic acid comprises an ORF encoding a viral capsid sequence. In some embodiments, the nucleic acid comprises a synthetic 5′ UTR in combination with a non-synthetic 3′ UTR.

In some embodiments, the UTR can also include at least one translation enhancer nucleic acid, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety for this purpose, and others known in the art. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation. In one non-limiting example, the TEE comprises the TEE sequence in the 5′-leader of the Gtx homeodomain protein. See, e.g., Chappell et al., PNAS. 2004. 101:9590-9594, incorporated herein by reference in its entirety for this purpose.

### Poly(A) Tails

Aspects of the present disclosure relate to methods of producing RNAs containing one or more polyA tails. A “polyA tail” is a region of mRNA that is downstream, e.g., directly downstream (i.e., 3′), from the open reading frame and/or the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

As used herein, “polyA-tailing efficiency” refers to the amount (e.g., expressed as a percentage) of mRNAs having polyA tail that are produced by an IVT reaction using an input DNA relative to the total number of mRNAs produced in the IVT reaction using the input DNA. The polyA-tailing efficiency of an IVT reaction may vary, for example depending upon the RNA polymerase used, amount or purity of input DNA used, etc. In some embodiments, the polyA-tailing efficiency of an IVT reaction is greater than 85%, 90%, 95%, or 99.9%. Methods of calculating polyA-tailing efficiency are known, for example by determining the amount of polyA tail-containing mRNA relative to total mRNA produced in an IVT reaction by column chromatography (e.g., oligo-dT chromatography).

In some embodiments, at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.9% of RNAs in an RNA composition produced by a method described herein comprise a polyA tail. In some embodiments, at least 85%, 86%, 87%, 88%, 89%, 90%, 91%, 92%, 93%, 94%, 95%, 96%, 97%, 98%, 99%, or 99.9% of each RNA in an RNA composition produced by a method described herein comprise a polyA tail. The efficiency (e.g., percentage of polyA tail-containing RNAs in an RNA composition may be measured i) after the IVT reaction and before purification, or ii) after the RNA composition has been purified (e.g., by chromatography, such as oligo-dT chromatography).

Unique polyA tail lengths provide certain advantages to the nucleic acids of the present disclosure. Generally, the length of a polyA tail, when present, is greater than 30 nucleotides in length. In another embodiment, the polyA tail is greater than 35 nucleotides in length (e.g., at least or greater than about 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides).

In some embodiments, the polyA tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the polyA tail can be 10, 20, 30, 40, 50, 60, 70, 80, 90, or 100% greater in length than the nucleic acid or feature thereof. The polyA tail can also be designed as a fraction of the nucleic acid to which it belongs. In this context, the polyA tail can be 10, 20, 30, 40, 50, 60, 70, 80, or 90% or more of the total length of the construct, a construct region or the total length of the construct minus the polyA tail. Further, engineered binding sites and conjugation of nucleic acids for PolyA-binding protein can enhance expression.

### EXAMPLES

### Example 1: Reductions in PCR Products Containing Sequence Errors Following Dual Nuclease Treatment

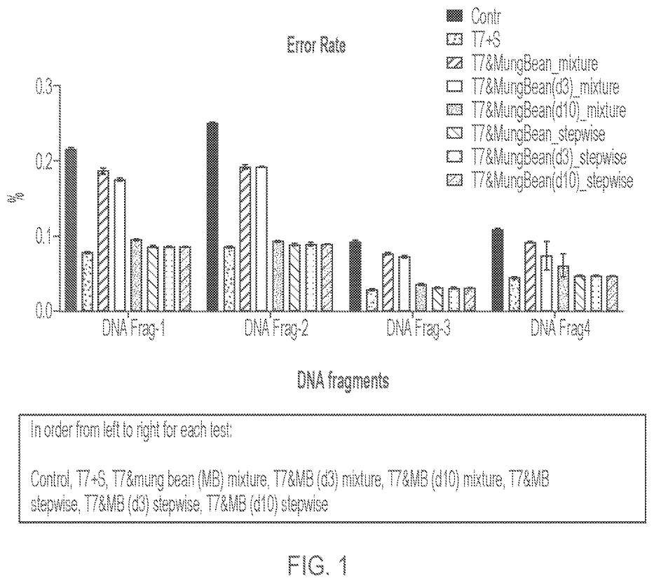

This example describes sample preparation methods to assess error removal efficiency in PCR products generated from DNA templates that were subjected to T7E1 and/or mung bean nuclease digestion. Error rates in re-assembly PCR products generated from DNA template samples purified using T7E1 and mung bean nuclease treatments prior to gene synthesis were calculated. Four DNA fragments (“DNA Frag-1,” “DNA Frag-2,” “DNA Frag-3,” and “DNA Frag-4” in FIG. 1) were diluted to 10 ng/μL in NEB2 buffer (50 mM NaCl, 10 mM Tris-HCl, 10 mM MgCl₂, 1 mM DTT) and duplexed. To generate T7E1-digested control samples, 20 μL of duplexed DNA in NEB2 was mixed with 2 μL of T7 endonuclease I (T7E1) (NEB) and incubated at 37° C. for 30 minutes. Then, T7E1-digested controls were cleaned up by SPRI purification using 20 μL of Cytvia beads followed by elution with 20 μL of water (“T+S” samples in FIG. 1). In parallel, T7E1 and mung bean nuclease dually digested samples were prepared using either “mixture” digestion, wherein T7E1 and mung bean endonuclease were co-incubated at 37° C. for 30 minutes, or “stepwise” digestion, wherein 20 μL of duplexed DNA in NEB2 buffer was mixed with T7E1 and incubated at 37° C. for 45 minutes followed by addition of mung bean nuclease (NEB) for an additional 30 minutes at 37° C. Dually digested samples underwent no clean-up step using SPRI purification. Dually digested samples, processed either through mixture or stepwise digestion, were prepared three ways using either 2 μL each of T7E1 and mung bean endonuclease (at a stock concentration of 10,000U/mL), using 2 μL each of T7E1 and a 1:3 dilution (“d3” in FIG. 1) of mung bean endonuclease, or 2 μL each of T7E1 and a 1:10 dilution (“d10” in FIG. 1) of mung bean endonuclease. Following digestion of DNA samples, 15 μL of reaction products were reassembled and amplified by PCR. PCR products were then cleaned up and submitted to next generation sequencing for error rate quantification. Analyses of error rates in digested samples showed that dually digested samples that were processed without a SPRI purification step exhibited similar error rates to samples digested with T7 alone and processed with a SPRI purification step. Additionally, under the conditions tested, at more concentrated dilutions of mung bean nuclease (e.g. dilutions using mung bean nuclease stock concentrations at 10,000U/mL and d3) the stepwise digestion conditions appeared to result in lower error rates relative to the same dilutions of mung bean nuclease used as a mixture (FIG. 1).

### Equivalents and Scope

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for processing a DNA, the method comprising:
preparing a sample of heteroduplex DNA, wherein at least one heteroduplex DNA in the sample comprises a mismatch DNA having one or more sequence errors, performing a dual nuclease digestion on the sample to produce a digested product by contacting the sample with T7E1 endonuclease to cleave the mismatch DNA at the sequence error site to produce one or more DNA fragments and contacting the sample with mung bean nuclease to degrade the DNA fragments, thereby producing a purified sample of heteroduplex DNA.

2. The method of claim 1, wherein the purified sample of heteroduplex DNA produced by the method has error-rate reductions of 15-60% relative to a comparable method performed without digestion with mung bean nuclease.

3. The method of claim 1, wherein the purified sample of heteroduplex DNA produced by the method has error-rate reductions of 20-30% relative to a comparable method performed without digestion with mung bean nuclease.

4. The method of any one of claims 1-3, wherein less than 5% of total nucleic acid in the purified sample of heteroduplex DNA is comprised of mismatched DNA and DNA fragments.

5. The method of any one of claims 1-4, wherein at least 99% of heteroduplex DNA has 100% base complementarity and wherein at least 99% of the heteroduplex DNA is full length.

6. The method of any one of claims 1-5, wherein a re-assembly PCR step is performed following T7E1 endonuclease and mung bean nuclease digestion on the digested product, thereby producing a purified sample of DNA template.

7. The method of claim 6, wherein a purification step is performed following re-assembly PCR.

8. The method of claim 7, wherein the purification step is a solid-phase reversible immobilization (SPRI) paramagnetic bead process.

9. The method of any one of claims 6-8, wherein a purification step is not performed between the T7E1 endonuclease and mung bean nuclease digestion and the re-assembly PCR.

10. The method of any one of claims 6-9, wherein the digested product is used in the re-assembly step at a maximum volume of 50 μL.

11. The method of any one of the preceding claims, wherein the endonuclease is T7E1.

12. The method of any one of the preceding claims, wherein the nuclease is wild type mung bean nuclease.

13. The method of any one of the preceding claims, wherein the sample is contacted with the T7E1 and mung bean nucleases at the same time.

14. The method of any one of the preceding claims, wherein the sample is contacted with T7E1 and mung bean nucleases at separate times.

13. The method of any one of the preceding claims, wherein the sample comprises 1:1 T7E1 endonuclease:mung bean nuclease.

14. The method of any one of the preceding claims, wherein the dual nuclease digestion step is performed at least two times.

17. The method of any one of the preceding claims, wherein the process is a commercial batch process.

18. The method of any one of the preceding claims, wherein the sequence error comprises a substitution, deletion, or insertion of between 1 and 10 nucleotides.

19. The method of any one of the preceding claims, further comprising producing mRNA with the purified sample of heteroduplex DNA.

20. A purified sample of DNA template, comprising a plurality of heteroduplex DNA, wherein at least 99% of the heteroduplex DNA has 100% base complementarity and wherein at least 99% of the heteroduplex DNA is full length.

21. A purified sample of DNA template, comprising a plurality of DNA template, wherein at least 99% of the DNA template has 100% base complementarity and wherein at least 99% of the DNA template is full length.

22. A composition comprising a heteroduplex DNA comprising a mismatch DNA having one or more sequence errors, T7E1 endonuclease, and mung bean nuclease.

23. A composition comprising a plurality of heteroduplex DNA, T7E1 endonuclease, and mung bean nuclease, wherein at least 90-100% of the heteroduplex DNA is full length.
